(19) United States
(12) Patent Application Publication
(10) Pub. No.: US 2025/0308687 A1
(43) Pub. Date: Oct. 2, 2025

(54) TREATMENT CENTER FOR CARRYING OUT MEDICAL INFUSIONS AND ASSOCIATED PROCEDURE

(71) Applicant: B. Braun Melsungen AG, Melsungen (DE)

(72) Inventor: Felix Kever, Kassel (DE)

(21) Appl. No.: 19/097,008

(22) Filed: Apr. 1, 2025

(30) Foreign Application Priority Data
Apr. 2, 2024 (EP) 24168053.7

(51) Int. Cl. G16H 40/63; A61M 5/142; G16H 10/60

(52) U.S. Cl. CPC G16H 40/63; A61M 5/142; G16H 10/60; A61M 2005/14208

(57) ABSTRACT

A treatment center for carrying out medical infusions includes a management system, an infusion pump system, and identification features labeled with verification codes. The management system includes a database that assigns each patient to a patient ID or case number. The infusion pumps each have a user interface with an input element for entering a verification code. The identification features can be body-worn features, such as patient wristbands, that are labeled with a patient verification code for temporary identification of a patient for a predetermined treatment period. Alternatively, the identification features can be bed locations that are each assigned to one of the infusion pumps and labeled with a bed location verification code. A patient or a bed location can be assigned to an infusion pump based on a verification code entered at the infusion pump. Each verification code can be an alphanumeric code comprising a number of digits.

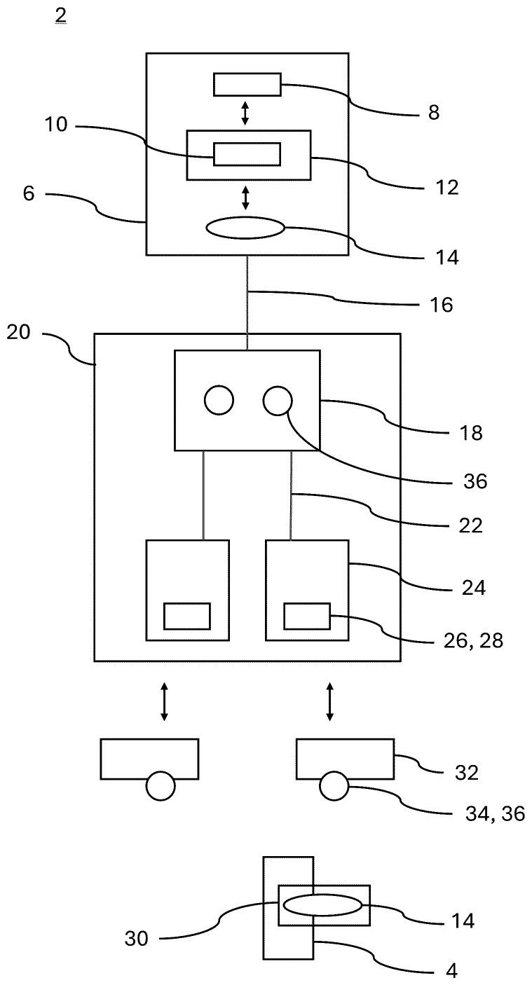

### CROSS-REFERENCE TO RELATED APPLICATION

This application claims priority to European Application No. 24168053.7, filed on Apr. 2, 2024, the content of which is incorporated by reference herein in its entirety.

### FIELD

The present disclosure relates to a treatment center for carrying out medical infusions. It also relates to a method for operating an infusion pump system.

### BACKGROUND

The secure assignment of medical devices to the respective patient is absolutely essential if there is more than one patient in the system context at the same time and the therapy, usage and operating data generated by the respective device is to be processed in other systems. Otherwise, anonymous device data would have to be retrospectively assigned to the correct patient or discarded.

A common procedure for infusion pumps is indirect patient assignment via the bed location: The higher-level Hospital Information System (HIS) or Electronic Medical Record (EMR) system maintains a list of bed locations to which patients from the patient list are temporarily assigned as part of a relational database. If the infusion pumps are now also assigned to a bed location and both assignment lists are kept up to date, it is possible to clearly assign a pump to a patient at any time.

If the pump is assigned to the bed location by transferring the bed location information from fixed rack systems with a fixed bed location assignment, this method works reliably and largely automatically. It is more difficult with pumps that are not used with rack systems but as “mobile” devices: Here, there is usually no technical solution for automatically assigning the current bed location; moreover, the devices are usually subject to quite a large spatial fluctuation in view of their small size and comparatively large number.

However, the treatment workflows in hospitals can also be problematic for indirect patient allocation: In operating theaters or intensive care units, entire rack systems are called in spontaneously (i.e. they are not stationary and must first be assigned to the new bed location), while in emergency situations patients have to be infused who are not (yet) assigned to a bed location—in the worst case, in the corridor.

Currently, the only option in this case is to manually assign the pump to the correct bed location before each infusion start—either via the local user interface, the user interface of the backend, or a smart device with scan function, which is connected to the backend and serves as its remote control.

One possible procedure is based on patient assignment via a selection process using the user interface of the pump (or medical device in general). For this purpose, the patient list (possibly only an extract, filtered by the care unit configured on the device) is first downloaded from the HIS. With suitable filtering, e.g. according to the first letters of the name or other criteria, the list can be shortened in such a way that it enables a practicable/user-friendly selection of the patient. In any case, the database query/filtering/selection required for a successful assignment requires some user interaction. In addition, there are data protection challenges due to the processing of a large number of sensitive personal data records on the medical device or—looking at it the other way round—due to the read access to the entire patient database provided by each individual infusion pump.

In any case, the assignment is related to a certain amount of configuration work, which, due to the relevance of the correct assignment in combination with the spatial mobility of the infusion pumps, must be carried out by the medical staff at the patient's bedside and is usually viewed very critically in view of the usual workload.

One task of the present disclosure is to provide a treatment center for performing medical infusions and an associated method for simple, reliable and scannerless patient assignment of infusion pumps or other medical devices to a patient and/or bed location, which is easy to implement and allows a high degree of flexibility in everyday clinical practice.

### SUMMARY

According to the present disclosure, said task is solved by a treatment center as described below.

Accordingly, a treatment center is planned for the implementation of medical infusions, comprising:
a. an administration system with a database for storing patient data, whereby each patient is assigned a patient ID or a case number,
b. an infusion pump system with a backend that is connected to the administration system and to a number of infusion pumps via data connections,
c. a plurality of infusion pumps, each with a user interface including an input element that enables the input of a short verification or identification code, which will be abbreviated herein as SVC.
d. a plurality of body-worn identification features, in particular patient wristbands, each labeled with a patient SVC, for the temporary, unique identification of patients for a predetermined treatment period, or
a plurality of bed locations each assigned to one of the infusion pumps, each labeled with a unique bed location SVC,
whereby the management system and/or the backend is designed to make an assignment between patient and infusion pump or between bedside and infusion pump using a patient SVC or bedside SVC entered at one of the infusion pumps,
where the respective SVC is an alphanumeric code comprising a number of digits,
where the management system contains a list of valid patient SVCs, each of which is temporarily assigned to a patient ID or a case number.

It is advantageous if the respective SVC has a smaller number of digits than an assigned patient ID. This means that the SVC is at least one digit and preferably several digits shorter than the assigned patient ID, which is preferably also an alphanumeric code. The respective SVC is particularly preferably a two- or three-digit alphanumeric code.

The tasks, features and advantages mentioned in relation to the device apply mutatis mutandis to the method and vice versa.

The concept of code-based patient assignment (CPA) described here solves the problem described above with the help of an alphanumeric Short Verification Code (SVC), which can be easily entered by the human user without technical aids and is ideally entered via a dedicated input element on the infusion pump. In particular, the input element can be a soft keyboard, i.e. an on-screen keyboard on a touchscreen of the infusion pump, which is preferably specially optimized for the SVC used. Even a three-digit alphanumeric code offers around 42,000 variants, which is still far too few for mapping the patients treated in a large hospital over time or the case numbers assigned to these patients.

For this reason, the CPA concept stipulates that the SVC printed on the patient wristband is linked to the permanently unique case number and patient ID stored in the HIS or EMR system (generally: in the administration system) over a defined time window. It is therefore based on a temporary and historicized assignment of SVC and patient ID.

For example, the SVC “B3X” is linked to the patient ID “08154711-1234” for a period of four weeks—starting with admission to hospital. To assign an infusion pump to this patient, this three-digit SVC simply needs to be entered on the pump, which is as easy as it is user-friendly if a touchscreen is available with the aid of a dedicated soft keyboard. The pump now sends the SVC to the HIS or EMR system via the backend of the infusion pump system and in return receives the currently linked patient ID and—optionally—the name, age and gender of the patient.

The latter metadata—in addition to the determined patient ID—is only displayed on the UI of the pump in order to provide the user with additional feedback on the correctness of the assignment in a simple manner. Immediately after the user's positive or negative confirmation, however, this data is deleted; only the patient ID is permanently stored in the pump, which is then also included in the data stream sent to the EMR system after the infusion is started.

The concept of code-based location assignment (CLA), which can be understood as a modification or variant of the CPA concept, also solves the problem described at the beginning with the help of an alphanumeric short verification code (SVC), which can be easily entered by the human user without technical aids and ideally entered via a dedicated soft keyboard on the infusion pump. A two-digit alphanumeric code already offers around 1,200 variants, which is sufficient for mapping the bed locations typically available in a hospital. The approximately 42,000 variants of a three-digit alphanumeric code would easily cover even the largest hospital in the world (Texas Medical Center, Houston) with around 9,200 beds.

There are theoretically three variants for using this code, two of which are preferable—depending on the architecture of the bed location assignment:

### Case A:

The location SVC is entered in/at the pump; in the infusion pump backend, the corresponding bed location designation is searched for in accordance with the hospital structure and, if successful, shown on the pump display for confirmation (including metadata). After confirmation, the assignment between pump and bed location is made in the backend and the bed location designation is transmitted to the downstream systems together with the infusion data.

### Case B:

The location SVC is entered in/on the pump; the infusion pump backend requests the corresponding bed location designation from the higher-level EMR system or HIS. If successful, this is shown on the pump display for confirmation. After confirmation, the received bed location designation is transmitted to the downstream systems together with the infusion data.

### Case C:

The location SVC is entered in/at the pump; this is transmitted 1:1 together with the infusion data to the downstream systems. Problem: The user receives no feedback on the basis of which he could check or at least assess the correctness of his entry. In this respect, this type of bed location allocation is relatively uncertain. For this reason, cases A and B are given priority in the following.

In addition, the following should be noted: Only the infusion pump system (i.e. the backend and/or the individual pump) “knows” which pumps are located at which bed positions.

The pump data sent to the management system contains the bed location as additional information so that the management system can assign it to the correct patient.

They also contain the ID of the pump, so that the management system theoretically also knows which pumps are currently at which bed location. However, this is not a necessary prerequisite for the challenge actually addressed by the present disclosure (assigning the pump data to the correct patient).

In this respect, the rule is that the backend manages and updates the assignment between bed locations and infusion pumps in accordance with the bed location SVCs.

### General CPA or CLA Requirements

The concepts described here are based on an existing network or data connection between the infusion pump, the backend of the infusion pump system and the HIS or EMR system (generally: the administration system).

### CPA- or CLA-Related Innovations

A fundamentally new feature is a historical patient-specific or bedside-specific, preferably alphanumeric Short Verification Code (SVC), which can be easily read from the patient wristband or bedside label and entered via the user interface of the infusion pump. With just three alphanumeric characters (Latin capital letters without “O” and digits from 0 to 9), the SVC offers around 42,000 code variants, which, in combination with a time-limited assignment, is sufficient to map all patients currently undergoing treatment or all bed locations, even in a large hospital. This means that there is no need for a scanner or other reading device (e.g. smart device) when assigning medical devices to patients.

In the case of CPA, there are the following structural requirements/boundary conditions and specific advantages:

### CPA-Related Innovations in the Infusion Pump System

An input option for the patient SVC on the infusion pump
An indirect database query via the backend of the infusion pump system based on the entered SVC
A display and confirmation dialog for the patient ID determined on the basis of the patient SVC

### CPA-Related Changes to the HIS or EMR System

A list of valid patient SVCs, each of which is temporarily assigned to a patient ID or a case number (which in turn is assigned to a patient ID).
A query option for the patient ID or case number as well as the name, age and gender of the patient based on the patient SVC.
Patient wristbands on which the currently assigned patient SVC is printed in addition to the patient ID.

### Advantages of the CPA System Concept:

No scanner, no smart device and no interaction with other systems is required for patient allocation. This makes implementation much easier and faster. The maintenance effort and the number of possible sources of error are also reduced.
The assignment is possible for individually used devices as well as for infusion pumps that are used in rack systems. It does not matter whether the rack systems are stationary or not.
All error possibilities of an indirect assignment via the bed location are eliminated without replacement (emergency patient does not yet have a bed location, assignment of patient< >bed location is delayed/is no longer up-to-date . . . ).
Assignment takes place directly at the infusion pump, but compared to existing solutions, the interaction is extremely simple and fast (entry of a preferably three-digit code from the patient wristband, confirmation of the assigned patient data).

In the case of CLA, there are the following structural requirements/boundary conditions and specific advantages:

### CLA-Related Innovations in the Infusion Pump System

An input option for the bed location SVC on the pump.
In case A: A database query of the pump to the component of the higher-level infusion pump backend responsible for managing the hospital structure (output of the bed location designation for the respective location SVC).
In case B: An indirect database query via the backend of the infusion pump system to the higher-level EMR system or HIS responsible for the bed location assignment (output of the bed location designation for the respective location SVC).
In case A: A display and confirmation dialog for the bed location determined on the basis of the location SVC, including display of bed location—related metadata from the hospital structure (e.g. room, care unit, building, etc.)
In case B: A display and confirmation dialog for the bed location determined on the basis of the location SVC

### CLA-Related Innovations in the Infusion Pump Backend

In case A: A list of valid and unique bed location SVCs, each of which is assigned to a bed location (“Point of Care”) in the Hospital Structure.
In case A: An internal system query option for the bed location designation based on the location SVC, including the return of associated metadata (e.g. room, care unit, building, etc.).
In case B: A query option with the higher-level EMR system or HIS for the bed location designation based on the location SVC

### CLA-Related Changes to the HIS or EMR System

In case A: n/a.
In case B: A list of valid and unique location SVCs, each of which is assigned to a bed location.
In case B: A query option for the bed location designation based on the bed location SVC.

### CLA-Related Innovations in the Hospital or Patient Room

Signs, stickers or similar at each bed location on which the bed location designation and the corresponding location or bed location SVC can be easily read.

### Advantages of the CLA System Concept:

Assignment takes place directly at the infusion pump, but compared to existing solutions, the interaction is extremely simple and fast (entry of a two- or three-digit code read at the bedside, confirmation of the assigned bedside, including metadata if necessary).
No scanner, no smart device and no interaction with other systems required. This makes implementation much easier and faster. The maintenance effort and the number of possible sources of error are also reduced.
The bed location assignment is possible for individually used devices as well as for infusion pumps that are used in rack systems. It does not matter whether the rack systems are stationary or not.
The concept covers bed location assignment within the infusion pump backend (case A) as well assignment in the higher-level EMR system or HIS (case B).

As a result, the device and method according to the present disclosure simple and scannerless patient or bed location assignment of infusion pumps or other medical devices—as a replacement both for the indirect (and in certain cases hardly realizable) traditional assignment via the bed location, as well as for the list selection on the device or other workarounds.

### BRIEF DESCRIPTION OF THE DRAWING FIGURE

Several embodiments of the present disclosure are explained in more detail below with reference to the accompanying FIGURE.

The FIGURE shows a highly simplified structural diagram of a treatment center for performing medical infusions.

### DETAILED DESCRIPTION

The medical treatment center 2 shown in a schematic overview in the FIGURE is designed to perform medical infusions on a variety of changing patients 4. A computerized management system 6, which may be a hospital information system (HIS) or an electronic medical record (EMR)-based system, includes a database in which treatment cases are stored, for example, sorted by case number 8. Each case number 8 is assigned to a patient 4 who can be identified by a unique patient identifier or patient ID 10. This is usually a longer character string or multi-digit combination of letters and/or numbers, specifically for example: 08154711-1234. The patient ID 10 is therefore comparatively long so that each patient 4 who visits the treatment center 2 can be clearly identified, even over a period of years. Each patient ID 10 is assigned a complete data set of patient data 12, which includes, for example, the patient's name, age and gender.

The length of the patient ID 10 makes error-free manual input into data-processing systems or devices difficult. On the other hand, a much shorter string of characters and/or digits is sufficient for a predetermined validity period of four weeks, for example, in order to uniquely identify the typical volume of patients in the treatment center 2. For this reason, the administration system 6 generates a short form for the patient ID 10 of a patient 4 currently undergoing treatment or intended for treatment, which uniquely identifies the patient for a specified validity period or a planned treatment period, for example a period of four weeks, alongside other patients 4 currently undergoing treatment. This so-called short identification code or short verification code (SVC), also known as patient SVC 14, is preferably a code consisting of a few digits, in particular an alphanumeric code, in which each of the digits is preferably selected from the entirety of the capital Latin letters from A to Z and the digits 0 to 9 or from a subset thereof (for example only letters or only digits). Due to the risk of confusion, it is advantageous not to differentiate between “O” and “0”. A three-digit code is particularly preferred, especially a three-digit alphanumeric code, which is easy for a human user to grasp and memorize for a short time and yet allows around 42,000 possible combinations under the aforementioned conditions. In order to avoid multiple assignment, the management system 6 can mark and/or segregate the currently assigned patient SVCs 14 accordingly, whereby they are released again after the validity period has expired.

The management system 6 transmits the patient SVC 14, possibly together with other patient data 12, to a label printer not shown or to another labeling system, so that as a result a patient wristband 30 to be worn by the patient 4 during the stay in the treatment facility 2 is labeled with the patient SVC 14 and possibly further information.

The management system 6 is connected to a backend 18 of an infusion pump system 20 via a (wired or wireless) data connection 16. In turn, a plurality of infusion pumps 24 can be connected to the backend 18 via corresponding (wired or wireless) data connections 22. The spatial arrangement of the infusion pumps 24 in the treatment center 2 can change over time, and depending on availability, a patient 4 can be assigned for an infusion treatment, at least in principle, to any free infusion pump 24 connected to the backend 18.

However, for safe operation tailored to individual patient needs with corresponding individual programming or dosage settings, it is necessary that the administration system 6 and/or the backend 18 knows the assignment between patient 4 and infusion pump 24, and/or that the infusion pump 24 “knows” the patient 4 and can, for example, download dosage instructions or the like assigned to it from the administration system 6. For this purpose, the respective infusion pump 24 has a user interface with an input option or an input element 26 for the patient SVC 14 of the currently connected patient 4. Advantageously, this is a graphical user interface (GUI) with a touch screen 28 on which a dedicated soft keyboard is implemented. Alternatively, however, it can also be a hardware keyboard or a hardware keypad with a separate display. Input via a keyboard assigned to the infusion pump 24 at another location in the vicinity is also conceivable.

A member of the treating medical staff reads the patient SVC 14 from the patient wristband 30 of the patient 4 to be connected or already connected to the infusion pump 24 and enters it via the input element 26. The infusion pump 24 now sends the patient SVC 24 (via the data connections 16, 22) to the administration system 6 via the backend 18 of the infusion pump system 20 and, in return, receives the currently linked patient ID 10 and—optionally—patient data 12 such as the name, age and gender of the patient 4 after a corresponding database query.

The latter metadata—in addition to the determined patient ID 10—is only displayed on the user interface of the infusion pump 24 in order to provide the user with additional feedback on the correctness of the assignment in a simple manner. Immediately after the user's positive or negative confirmation, however, this data is deleted; only the positively verified patient ID 10 is stored in the infusion pump 24 for longer (in this context, this means at least for the intended treatment duration), which is then also contained in the data stream sent to the management system 6 after the start of the infusion.

As a result, the uncomplicated input of the patient SVC 14 read from the patient wristband 30 directly at the infusion pump 24 enables a correct assignment between patient 4 and infusion pump 24 for the respective treatment (infusion). This is possible without knowing the exact location of the infusion pump 24. In comparison to the permanent assignment between patient ID 10, case number 8 and patient data 12, this assignment between patient 4 and infusion pump 24 is temporary and is updated in the management system 6 correspondingly frequently in the event of a change—as is the patient SVC 14, which is valid for a few weeks at most.

In a modification of the concept described, the infusion pumps 24 are set up at dedicated treatment locations, in particular bed locations 32. For the sake of simplicity, the term bed locations 32 is used in the following, although the more general case of a fixed (immobile) treatment location is always included. The respective bed location 32, to which an associated infusion pump 24 is connected, is provided with a label 34 or marking which contains a bed location designation or bed location description in plain text which is unique within the treatment center 2 and a uniquely assigned bed location SVC 36 (more generally: treatment location SVC or location SVC) as an abbreviated version thereof.

The same applies to bed location SVC 36 as to patient SVC 14, so that, for example, a three-digit alphanumeric code consisting of Latin capital letters from A to Z and numbers 0 to 9 (due to the risk of confusion, “O” and “0” are used synonymously) generates around 42,000 possibilities for a unique bed location designation—more than enough even for the very largest hospitals.

It is assumed that the scheduled, basic assignment between bed location 32 and infusion pump 24 is known in the backend 18 of the infusion pump system 20, i.e. is stored there, preferably also the bed location description.

If a patient 4 has been assigned to a free bed location 32 by a member of the medical staff or other user, the user reads the bed location SVC 36 from the associated label 34 and enters it via an input element 26 on the infusion pump 24. As already described in connection with the patient SVC 14, this may preferably be a soft keyboard as part of a graphical user interface or alternatively a hardware keypad.

The bed location SVC 36 entered at the infusion pump 24 is transmitted to the backend 18 via the data connection 22. In the backend 18, the corresponding bed location designation is searched for in accordance with the room layout plan or hospital structure plan of the treatment center 2 by means of a database query and, if successful, is shown on the display of the infusion pump 24 for confirmation (preferably including metadata). After confirmation at the input element 26 of the infusion pump 24, the specific assignment between infusion pump 24 and bed location 32 is made binding in the backend 18 and the bed location designation is transmitted to the downstream systems together with the infusion data.

In an alternative variant of this, the scheduled assignment between bed location 32 and infusion pump 24 is stored in the management system 6, and the backend 18 queries the information there. If successful, this is shown on the display of the infusion pump 24 for confirmation. Here too, after confirmation at the input element 26 of the infusion pump 24, the received bed location designation is transmitted to the downstream systems together with the infusion data.

It is also conceivable that only the bed location SVC 36 is entered at the infusion pump 24 and used directly without confirmation, i.e. transmitted together with the infusion data to the downstream systems. However, the user then receives no feedback on the basis of which he could check the correctness of his input. In this respect, this type of bed location assignment is not as secure as the variants described above.

In principle, it is possible to implement the structures and procedures based on patient SVC 14 and bed location SVC 36 on their own or, alternatively, to combine them.

Instead of a wristband, a neckband or an ankle band or another body-worn identification band or identification feature that cannot be removed without destroying it, such as an item of clothing, can also be used.

The concepts described can be used not only for infusion pumps, but in principle for any medical devices that allow patient-specific settings.

### LIST OF REFERENCE SYMBOLS

2 Treatment center
4 Patient
6 Management system
8 Case number
10 Patient ID
12 Patient data
14 Patient SVC
16 Data connection
18 Backend
20 Infusion pump system
22 Data connection
24 Infusion pump
26 Input element
28 Touch screen
30 Patient wristband
32 Bed location
34 Label
36 Bed location SVC

1. A treatment center for carrying out medical infusions for patients, the treatment center comprising:
a. a management system with a database for storing patient data, each patient being assigned a patient ID or a case number;
b. an infusion pump system with a backend connected to the management system via data connections;
c. a plurality of infusion pumps connected to the backend via data connections, each infusion pump comprising a user interface with an input element; and
d. a plurality of identification features, each identification feature assigned to a short verification code that is unique,
each input element being operable to input a short verification code,
the management system and/or the backend being configured to assign an infusion pump to a patient or to a bed location based on a short verification code that is input into the infusion pump, and
the short verification code comprising an alphanumeric code comprising a number of digits.

2. The treatment center according to claim 1, wherein:
the plurality of identification features comprise a plurality of body-worn identification features,
the short verification code comprises a patient verification code, and
the management system and/or the backend is configured to assign the infusion pump to a patient based on the patient verification code that is input into the infusion pump.

3. The treatment center according to claim 2, wherein the plurality of body-worn identification features comprise patient wristbands.

4. The treatment center according to claim 2, wherein the management system contains a list of the patient verification codes, each patient verification code being temporarily assigned to a patient ID or a case number.

5. The treatment center according to claim 2, wherein each patient verification code has a smaller number of digits than the patient ID assigned to said patient verification code.

6. The treatment center according to claim 2, wherein the patient verification code has no more than three digits.

7. The treatment center according to claim 2, wherein the management system is adapted to generate the patient verification code and transmit the patient verification code together with other patient data to a label printer or a labeling system to label one of the body-worn identification features.

8. The treatment center according to claim 2 wherein the management system manages and updates assignments of infusion pumps to patients according to the patient verification codes.

9. The treatment center according to claim 1, wherein:
the plurality of identification features comprise a plurality of bed locations,
the short verification code comprises a bed location verification code, and
the management system and/or the backend is configured to assign the infusion pump to a bed location based on the bed location verification code that is input into the infusion pump.

10. The treatment center according to claim 9, wherein the management system comprises a list of bed location verification codes, each assigned to a bed location of the treatment center.

11. The treatment center according to claim 9, wherein the backend manages and updates assignments of infusion pumps to bed locations according to the bed location verification codes.

12. The treatment center according to claim 9, wherein the management system manages and updates assignments of infusion pumps to bed locations according to the bed location verification codes.

13. The treatment center according to claim 1, wherein each user interface comprises a graphical user interface with a touch screen forming the input element.

14. The treatment center according to claim 1, wherein the management system is adapted to send a set of patient data or a bed location description to one of the plurality of infusion pumps for display and confirmation by a user upon receipt of a short verification code that is input into said one of the plurality of infusion pumps.

15. A method for operating the infusion pump system in the treatment center according to claim 1, the method comprising the steps of:
entering a short verification code into one of the plurality of infusion pumps; and
assigning said one of the plurality of infusion pumps to a patient or to a bed location based on the short verification code that is entered,
wherein the management system and/or the backend receives the short verification code that is entered and assigns said one of the plurality of infusion pumps to the patient or to the bed location based on the short verification code.
